(19) United States
(12) Patent Application Publication
(10) Pub. No.: US 2023/0157931 A1
(43) Pub. Date: May 25, 2023

(54) TOPICAL COMPOSITION BASED ON POROUS PARTICLES AND A CROSSPOLYMER COMPRISING ADIPIC ACID AND NEOPENTYLGLYCOL

(71) Applicant: Conopco, Inc., d/b/a UNILEVER, Englewood Cliffs, NJ (US)

(72) Inventors: Xiujuan CAO, Nanjing (CN); Lin WANG, Shanghai (CN)

(73) Assignee: Conopco, Inc., d/b/a UNILEVER, Englewood Cliffs, NJ (US)

(21) Appl. No.: 17/912,988

(22) PCT Filed: Mar. 12, 2021
(86) PCT No.: PCT/EP2021/056284

§ 371 Date: Sep. 20, 2022

(30) Foreign Application Priority Data
Mar. 31, 2020 (CN) PCT/CN2020/082322
May 13, 2020 (EP) 20174419.0

(51) Int. Cl. A61K 8/02; A61K 8/25; A61K 8/85; A61Q 19/08

(52) U.S. Cl. CPC A61K 8/0279; A61K 8/25; A61K 8/85; A61Q 19/08

(57) ABSTRACT

The present invention relates to a personal care composition. In particular, the personal care composition is directed to providing enhanced skin appearance like improved blurring. The present invention provides a personal care composition comprising: a) a porous particle; and b) a crosspolymer comprising adipic acid and neopentyl glycol.

### FIELD OF THE INVENTION

The present invention relates to a personal care composition. In particular, the personal care composition is directed to providing enhanced skin appearance like improved blurring.

### BACKGROUND OF THE INVENTION

Ageing brings many changes to the appearance of skin. Of particular concern to individuals wishing to maintain a youthful appearance, is the reduction or elimination of skin imperfections such as wrinkles, age spots or general unevenness of skin tone. Another problem is pore visibility. With the age the pore of the skin starts appearing in a distinct manner. Because of this the optical properties of the skin get negatively affected. Another preferred skin attribute is that of reduced shine. A shiny skin is indicative of oiliness which is an attribute disliked by many consumers. Most people prefer a matte appearance of their skin.

There has been considerable effort by the cosmetics industry to provide compositions which can mask or at least reduce skin imperfections. Often this is achieved by using materials such as talc, silica, kaolin and other inorganic particulates. These inorganic particulates achieve a matte effect due to their optical properties.

There are many ways people have developed to make skin imperfections less visible. Hence making the skin looks younger and better.

WO15178007 (SHISEIDO CO LTD, 2015) discloses sunscreen products that do not require the use of organic, ultraviolet-absorbing agents. Effective protection of the skin from ultraviolet light is attained by increasing the amounts of titanium dioxide and zinc oxide. To mask the excessive whiteness that would otherwise result from the oxides, the sunscreen products contain multilayer-type encapsulations containing pigments.

WO17023691 (LOREAL, 2017) describes a cosmetic composition comprising (a) about 10 percent to about 20 percent by weight, based upon the total weight of the composition of a film forming agent; (b) about 1 percent to about 25 percent by weight, based upon the total weight of the composition of a particulate material; (c) two or more thickening agents; and (d) water. When applied to the skin, the composition allows for immediate tightening of the skin while decreasing the visibility of fine lines and deep wrinkles.

The present inventors have found that as light scattering from the pores are also responsible for the appearance of the skin, controlling this scattering by application of a cosmetic composition is one way of making the skin looking younger and better.

### SUMMARY OF THE INVENTION

In a first aspect, the present invention provides a personal care composition comprising
a) a porous particle; and
b) a crosspolymer comprising adipic acid and neopentyl glycol, wherein the crosspolymer comprising adipic acid and neopentyl glycol is present in the range of 5% to 70% by weight of the composition and the porous particle is different from the crosspolymer.

In a second aspect, the present invention provides a method of improving the appearance of skin by providing improved blurring comprising the step of applying a composition of the present invention on the desired skin surface.

In a third aspect, the present invention provides use of composition of the present invention for reducing the appearance of fine lines, wrinkles, pores and/or blemish spots; evening skin tone, or a combination thereof on the desired skin surface.

All other aspects of the present invention will more readily become apparent upon considering the detailed description and examples which follow.

### DETAILED DESCRIPTION OF THE INVENTION

These and other aspects, features and advantages will become apparent to those of ordinary skill in the art from a reading of the following detailed description and the appended claims. For the avoidance of doubt, any feature of one aspect of the present invention may be utilized in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated. Except in the operating and comparative examples, or where otherwise explicitly indicated, all numbers in this description and claims indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated.

The disclosure of the invention as found herein is to be considered to cover all embodiments as found in the claims as being multiply dependent upon each other irrespective of the fact that claims may be found without multiple dependency or redundancy.

Where a feature is disclosed with respect to a particular aspect of the invention (for example a composition of the invention), such disclosure is also to be considered to apply to any other aspect of the invention (for example a method of the invention) mutatis mutandis.

By “A Personal Care Composition” as used herein, is meant to include a composition for topical application to the skin of mammals, especially humans. Such a composition may be generally classified as leave-on or rinse off but is preferably of the leave on type. The composition is formulated into a product which is applied to a human body specifically for improving appearance but may also be capable of providing cleansing, odor control or general aesthetics. The composition of the present invention can be in the form of a liquid, lotion, cream, foam, scrub, gel, or toner, or applied with an implement or via a face mask or a pad. Non-limiting examples of such compositions include leave-on skin lotions, creams, antiperspirants, deodorants, lipsticks, foundations, mascara, sunless tanners and sunscreen lotions. The composition of the present invention is preferably a leave-on composition. “Skin” as used herein is meant to include skin on the face and body (e.g., neck, chest, back, arms, underarms, hands, legs, buttocks and scalp) and especially to the sun exposed parts thereof.

The present invention provides a personal care composition comprising:
a) a porous particle; and
b) a crosspolymer comprising adipic acid and neopentyl glycol.

“Porous particle” as used herein refers to a particle with distributed voids throughout the entire volume of the particle. The voids can be individual or connected by small size openings, similar to pore openings that separate larger spaces.

“Specific surface area” as used herein typically refers to specific surface area determined according to Brunauer-Emmett-Teller method. The value of the specific surface area may be measured by following ASTM standard D 3663-78.

“Diameter” as used herein typically refers to particle diameter in non-aggregated state unless otherwise stated. For polydisperse samples having particulate with diameter no greater than 1 μm, diameter means the z-average diameter measured, for example, using dynamic light scattering (see international standard ISO 13321) with an instrument such as a Zetasizer Nano™ (Malvern Instruments Ltd, UK) unless otherwise stated. For polydisperse samples having particulate with diameter no less than 1 μm, diameter means the apparent volume median diameter (D50, also known as x50 or sometimes d(0.5)) of the particles measurable for example, by laser diffraction using a system (such as a Mastersizer™ 2000 available from Malvern Instruments Ltd) meeting the requirements set out in ISO 13320 unless otherwise stated.

The requirement for porous particle of the present invention is that the porous particle has the capability of absorbing large amounts of oils. The porous particle as used in the composition of the present invention has an oil absorption value preferably in the range from 50 g/100 g to 500 g/100 g. More preferably the porous particle has an oil absorption value of in the range from 75 g/100 g to 400 g/100 g, furthermore preferably from 100 g/100 g to 300 g/100 g and most preferably 120 g/100 g to 200 g/100 g. The oil absorption value typically refers to the values measured in conformity with ASTM Method D281-84.

The porous particle as per the invention has high pore volume to ensure the capability of oil absorption. Preferably, the pore volume value of the porous particle is greater than 0.04 m³/g, more preferably greater than 0.5 m³/g and most preferably greater than 0.8 m³/g. In other words, the pore volume value of the porous particle is preferably in the range from 0.05 m³/g to 5 m³/g, more preferably from 0.1 m³/g to 3 m³/g, further more preferably from 0.5 m³/g to 3 m³/g, most preferably from 0.5 m³/g to 2 m³/g. The pore volume value typically refers to the values measured in conformity with ASTM D4222-03.

The surface area of said porous particle is in the range from 50 m²/g to 500 m²/g, more preferably from 100 m²/g to 500 m²/g, furthermore preferably from 150 m²/g to 500 m²/g, even more preferably from 200 m²/g to 500 m²/g and most preferably from 220 m²/g to 400 m²/g.

The porous particle as per the invention preferably selected from porous silica, cellulose, acrylic polymer, nylon and ethylene/methacrylate copolymer hollow sphere coated with porous silica or a mixture thereof. Most preferably the porous particle is porous silica.

Preferably, the porous particle is present in the composition in amount of from 0.1 to 10% by weight of the composition, more preferably from 1 to 8%, furthermore preferably from 1 to 5%, most preferably from 1 to 3% by weight of the composition.

The composition of the present invention also comprises a crosspolymer comprising adipic acid and neopentyl glycol.

“Crosspolymer” as used herein refers to co-polymer which is made up of repeating units of one monomer or several monomers crosslinked with another monomer containing preferably at least 3 functional groups which is known as crosslinker. Different from non-crosslinked polymer, crosspolymer forms three-dimensional structure and hence exhibits good resistance to heat, solvent, wear, etc. Crosspolymers in general has been widely used in cosmetic products, as functional particles, film formers, thickeners, due to its unique chemical structure and excellent performance.

The crosspolymer of the present invention is a crosspolymer comprising adipic acid and neopentyl glycol. The crosspolymer used in the present invention preferably crosslinked with isopropyltriethylsilane. A particular example of a suitable and most preferred crosspolymer is adipic acid and neopentyl glycol crosslinked with isopropyltriethylsilane blended with a copolymer of vinyl pyrrolidone and vinyl acetate (INCI name adipic acid/neopentyl glycol crosspolymer).

The crosspolymer comprising adipic acid and neopentyl glycol preferably present in the range of 1% to 80% by weight, more preferably 5% to 70% by weight, furthermore preferably 7% to 60% by weight, even more preferably 8% to 50%, still even more preferably 12 to 42% by weight, and most preferably 18 to 35% by weight of the composition. The above weight percentage is the percentage of crosspolymer per se in the composition. In case the crosspolymer is added as a dispersion then the above percentage has to be construed accordingly.

For sake of clarity, the porous particle is different from the crosspolymer. Preferably, the weight ratio of the crosspolymer to the porous particle is in the range of 200:1 to 1:2, more preferably 100:1 to 1:1, even more preferably 60:1 to 2:1, still even more preferably 40:1 to 4:1 and most preferably 20:1 to 8:1.

The composition may optionally additionally comprise a silicone elastomer. The silicone elastomer used in the present invention is preferably powder of silicone elastomer. It is highly preferred that the silicone elastomer is cross-linked. Preferred silicone elastomers are organo-polysiloxanes available under the INCI names of dimethicone/vinyl dimethicone crosspolymer, dimethicone crosspolymer and Polysilicone-11®. More preferably the silicone elastomer is dimethicone/vinyl dimethicone crosspolymer.

Typically, the average diameter of the silicone elastomer is from 0.2 to 50 microns, more preferably from 0.5 to 20 microns, even more preferably from 0.8 to 10 microns, and still even more preferably from 1.5 to 6 microns.

The silicone elastomer is preferably present in amount of 0.5 to 20%, more preferably 1 to 15%, even more preferably from 2 to 6%, still even more preferably from 4.5 to 9% by weight of the composition.

The composition preferably additionally comprises one or more organic sunscreens. A wide variety of organic sunscreen is suitable for use in combination with the essential ingredients of this invention. Suitable UV-A/UV-B sunscreen include, 2-hydroxy-4-methoxybenzophenone, octyldimethyl p-aminobenzoic acid, digalloyltrioleate, 2,2-dihydroxy-4-methoxybenzophenone, ethyl-4-(bis(hydroxypropyl)) aminobenzoate, 2-ethylhexyl-2-cyano-3,3-diphenylacrylate, 2-ethylhexylsalicylate, glyceryl p-aminobenzoate, 3,3,5-trimethylcyclohexylsalicylate, methylanthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, 2-ethylhexyl-p-methoxycinnamate, butyl methoxydibenzoylmethane, 2-hydroxy-4-methoxybenzophenone, octyldimethyl-p-aminobenzoic acid and mixtures thereof. The most suitable organic sunscreens are 2-ethylhexyl-p-methoxycinnamate, butylmethoxydibenzoylmethane or a mixture thereof.

A safe and effective amount of organic sunscreen may be used in the compositions useful in the subject invention. The composition preferably comprises from 0.1% to 10%, more preferably from 0.1% to 5%, of organic sunscreen.

The composition of the invention preferably comprises a skin lightening agent. Vitamin B3 compounds (including derivatives of vitamin B3) e.g. niacin, nicotinic acid or niacinamide are the preferred skin lightening agent as per the invention, most preferred being niacinamide. Vitamin B3 compounds, when used, are preferably present in an amount in the range of 0.1 to 10%, more preferably 0.2 to 5% by weight of the composition.

The composition may comprise other beneficial skin care actives like retinol, retinyl esters, resorcinol, allantoin, ubiquinone, conjugated linoleic acid, 12-hydroxystearic acid or derivatives thereof. Of these the most preferred ones for inclusion in the composition of the invention are anti-aging actives like retinol or retinyl esters.

Compositions of the present invention will also include a cosmetically acceptable carrier which is a water and oil emulsion, which in certain embodiments may be water-in-oil emulsion. Preferred emulsions, however, are the oil-in-water variety. Preferred hydrophobic material for use in the oil phase of such emulsions includes emollients such as fats, oils, fatty alcohols, fatty acids, soaps, silicone oils, synthetic esters and/or hydrocarbons.

Silicones may be divided into the volatile and nonvolatile variety. Volatile silicone oils (if used) are preferably chosen from cyclic (cyclomethicone) or linear polydimethylsiloxanes containing from 3 to 9, preferably from 4 to 5, silicon atoms.

Nonvolatile silicones useful as an emollient material include polyalkyl siloxanes, polyalkylaryl siloxanes and polyether siloxane copolymers. The essentially nonvolatile polyalkyl siloxanes useful herein include, for example, polydimethyl siloxanes with viscosities of from about 5×10⁻⁶ to 0.1 m²/s at 25° C. Among the preferred nonvolatile emollients useful in the present compositions are the polydimethyl siloxanes having viscosities from about 1×10⁻⁵ to about 4×10⁻⁴ m²/s at 25° C.

Specific examples of non-silicone emollients include stearyl alcohol, glyceryl monoricinoleate, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, eicosanyl alcohol, behenyl alcohol, cetyl palmitate, silicone oils such as dimethylpolysiloxane, di-n-butyl sebacate, isopropyl myristate, isopropyl palmitate, isopropyl stearate, butyl stearate, polyethylene glycol, triethylene glycol, lanolin, cocoa butter, corn oil, cotton seed oil, olive oil, palm kernel oil, rape seed oil, safflower seed oil, evening primrose oil, soybean oil, sunflower seed oil, avocado oil, sesame seed oil, coconut oil, arachis oil, castor oil, acetylated lanolin alcohols, petroleum jelly, mineral oil, butyl myristate, isostearic acid, palmitic acid, isopropyl linoleate, lauryl lactate, myristyl lactate, decyl oleate, myristyl myristate, and mixtures thereof.

Among the ester emollients are:
a) Alkenyl or alkyl esters of fatty acids having 10 to 20 carbon atoms. Examples thereof include isoarachidyl neopentanoate, isodecyl neopentanoate, isononyl isonanoate, cetyl ricinoleate, oleyl myristate, oleyl stearate, and oleyl oleate;
b) Ether-esters such as fatty acid esters of ethoxylated fatty alcohols;
c) Polyhydric alcohol esters. Butylene glycol, ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters. Particularly useful are pentaerythritol, trimethylolpropane and neopentyl glycol esters of C₁-C₃₀ alcohols. An Example is pentaerythrityl tetraethylhexanoate;
d) Wax esters such as beeswax, spermaceti wax and tribehenin wax;
e) Sterols esters, of which cholesterol fatty acid esters are examples thereof;
f) Sugar ester of fatty acids such as sucrose polybehenate and sucrose polycottonseedate; or
g) mixtures of two or more of the foregoing (a) to (f).

Of particular use also are the C₁₂₋₁₅ alkyl benzoate esters sold under the Finsolv® brand.

Hydrocarbons which are suitable emollients include petrolatum, mineral oil, C₁₁-C₁₃ isoparaffins, polyalphaolefins, isohexadecane or a mixture thereof.

Amounts of water in the carrier may, for example, range from 1 to 99%, more preferably from 5 to 90%, even more preferably from 35 to 80%, optimally between 40 and 70% by weight of the personal care composition.

Other materials which can be included in the cosmetically acceptable carrier include solvents, humectants, thickeners and powders. Examples of each of these types of material, which can be used singly or as mixtures, are as follows:

Solvents include ethyl alcohol, isopropanol, acetone, ethylene glycol monoethyl ether, diethylene glycol monobutyl ether, diethylene glycol monoethyl ether and mixtures thereof.

Humectants include those of the polyhydric alcohol-type. Typical polyhydric alcohols include polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, isoprene glycol, 1,2,6-hexanetriol, glycerol, ethoxylated glycerol, propoxylated glycerol and mixtures thereof. The amount of humectant may range, for example, anywhere from 0.5 to 50%, more preferably between 1 and 15% by weight of the composition. Most preferred is glycerol (also known as glycerin). Amounts of glycerin may range, for example, from 0.5% to 50%, more preferably from 1 to 35%, optimally from 2 to 15% by weight of the composition.

A variety of thickening agents may be included in the compositions. Illustrative but not limiting are stearic acid, Acrylamide/Sodium Acryloyldimethyltaurate Copolymer (Aristoflex® AVC), Hydroxyethyl Acrylate/Sodium Acryloyldimethyltaurate Copolymer, Aluminum Starch Octenyl Succinate, Polyacrylates (such as Carbomers including Carbopol® 980, Carbopol® 1342, Pemulen TR-2® and the Ultrez® thickeners), Polysaccharides (including xanthan gum, guar gum, pectin, carageenan and sclerotium gums), celluloses (including carboxymethyl cellulose, ethyl cellulose, hydroxyethyl cellulose and methyl hydroxymethyl cellulose), minerals (including talc, silica, alumina, mica and clays, the latter being represented by bentonites, hectorites and attapulgites), magnesium aluminum silicate and mixtures thereof. Amounts of the thickeners may range, for example, from 0.05 to 10%, more preferably from 0.3 to 2% by weight of the composition.

Powders include chalk, talc, Fullers earth, kaolin, starch, gums, colloidal silica sodium polyacrylate, tetraalkyl and/or trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose and ethylene glycol monostearate.

The personal care composition of this invention is preferably a skin care composition. More preferably, the composition is preferably an antiperspirant composition or a face (except eye lids and lips) care composition. The skin care composition refers to a composition suitable for topical application to human skin, including leave-on and wash-off products. Preferably the term encompasses a fluid or liquid, and particularly a moisturizer rather than a make-up product. Most preferred are leave-on compositions. The term “leave-on” as used with reference to compositions herein means a composition that is applied to or rubbed on the skin and left thereon. The term “wash-off” as used with reference to compositions herein means a skin cleanser that is applied to or rubbed on the skin and rinsed off substantially immediately subsequent to application. The term “skin” as used herein includes the skin on the face (except eye lids and lips), neck, chest, abdomen, back, arms, under arms, hands, and legs. Preferably the term “skin” includes the skin on the face (except eye lids and lips) and under arms. More preferably it means skin on the face other than lips and eyelids.

The composition can be formulated in any known format, more preferred formats being creams or lotions.

Packaging for the composition of this invention can be a jar or tube as well as any other formats typically seen for cosmetic, cream, washing and lotion type products. The compositions may be applied topically and preferably 1-4 milligrams of composition is applied per square centimeter of skin.

The composition of the invention preferably delivers a cosmetic benefit to the skin of an individual to which it is topically applied. Examples of cosmetic benefits include reducing the appearance of fine lines, wrinkles, pores and/or blemish spots; evening skin tone, long lasting optical effect or a combination thereof on the desired skin surface.

The present invention also provides a method of improving the appearance of skin by blurring the imperfections of the skin comprising the steps of applying a composition as per the present invention on the desired skin surface.

The present invention further provides an use of a of the present invention for blurring benefit.

The Use of the composition as mentioned above wherein the blurring benefit is provided for reducing the appearance of fine lines, wrinkles, pores and or blemish spots on the skin.

The following examples are provided to facilitate an understanding of the invention. The examples are not intended to limit the scope of the claims.

### EXAMPLES

### Materials

TABLE 1

| | | | Surface | Pore | Oil |
|---|---|---|---|---|---|
| | | | area | Volume | absorption |
| Trade name | INCI name | Supplier | (m³/g) | (m²/g) | (g/100 g) |
| Halosphere N | Cross polymer of | DermaTech | NA | NA | NA |
| | adipic acid and | | | | |
| | neopentyl glycol | | | | |
| VALVANCE | Silica | DSM | 232 | 1.1 | 150 |
| Touch 210 | | | | | |
| MSS-500/3N | Silica | KOBO | 11 | 0.015 | 32 |

### Example 1: Effect of the Combination of Porous Particle and Crosspolymer of Adipic Acid and Neopentyl Glycol on the Blurring Efficacy

A series of skin care compositions were formulated as shown in Table 1.

TABLE 2

| | Examples | | | |
|---|---|---|---|---|
| Ingredient (wt %) | A | B | C | 1 |
| Halosphere N ^{a} | 50 | 0 | 50 | 50 |
| VALVANCE Touch 210 ^{b} | 0 | 2 | 0 | 2 |
| MSS-500/3N ^{c} | 0 | 0 | 2 | 0 |
| TWEEN 20 | 1 | 1 | 1 | 1 |
| Glycerin | 2 | 2 | 2 | 2 |
| Cyclopentasiloxane (D5) | 12 | 12 | 12 | 12 |
| Simulgel ® EG | 1.5 | 1.5 | 1.5 | 1.5 |
| Preservative | 0.5 | 0.5 | 0.5 | 0.5 |
| Water | To 100 | To 100 | To 100 | To 100 |

a: Halosphere N contains about 47% of Cross polymer of adipic acid and neopentyl glycol

b: VALVANCE Touch 210 contains 100% of silica (porous)

c: MSS-500/3N contains 100% of silica (solid, not porous particle)

The performance of the personal care compositions in Table 1 above was measured using the procedure as given below:

The image analysis algorithm was developed to extract the contrast between the pores and the background of BSP. The parameter of pore CWA, which calculates the contrast per image area for the pores, was used to measure and quantify the pore level. The simplest quantitative definition of contrast is the light intensity difference between a region of interest and its surroundings:

$$C = ( {L}_{b} - {L}_{f} )$$

Where C is the contrast, L_{f} is the light intensity (RGB value) of the pore, and L_{b} is the light intensity (RGB value) of the background. The higher pore CWA, the more visible pores. Thus, the pore CWA reduction is given by the following equation. The higher percentage of pore reduction, the better product efficacy.

$$\% Pore CWA reduction = \frac{Pore {CWA}_{baseline} - {CWA}_{product}}{Pore {CWA}_{baseline}} \times 100 \%$$

The commercial deep pore version of BioSkin plates (BSP) procured from Beaulax Co. Ltd., Japan (Code 10AN) were used as the in vitro substrate mimicking the appearance of human skin. The code 10AN representing sufficiently big pore size distribution. The above prepared formulations were then applied on this BSP with dosage of 2 mg/cm². It was then gently spread with finger cot, and then dried for 30 minutes at ambient temperature (˜25 C), prior to taking images.

One image was taken before applying the formulations, denoted as “bef”. Another image was taken after applying the respective formulations, denoted as “aft”. The “% change from baseline”, calculated as “(bef−aft)/bef*100%”, which is also % pore Contrast weight average (CWA) reduction as for the attributes of pore CWA. Which is nothing but denotes the blurring efficacy of a formulation. The higher the pore CWA reduction, the better the formulation is in delivering the blurring benefit.

The results are summarized below in Table 2.

TABLE 3

| Example | Pore CWA | |
|---|---|---|
| No | reduction (%) | SD |
| A | 19.0 | 4.0 |
| B | −1.7 | 3.8 |
| C | 20.3 | 4.5 |
| 1 | 27.6 | 1.5 |

From the data it is evident that the composition of Example 1 (both the crosspolymer and porous particle) provides much higher pore CWA reduction when compared with Example A (crosspolymer alone) and Example B (porous particle alone). The combination of the crosspolymer and porous particle as per the invention provides synergistic benefit in pore CWA reduction and in turn blurring efficacy of the composition. Another Example C with different silica particle does not provide as good benefit as Example 1.

1. A personal care composition comprising:
a) a porous particle; and
b) a crosspolymer comprising adipic acid and neopentyl glycol,
wherein the crosspolymer comprising adipic acid and neopentyl glycol is present in the range of 5% to 70% by weight of the composition, wherein the porous particle is different from the crosspolymer, and further wherein the porous particle has a pore volume greater than 0.04 m³/g.

2. The personal care composition as claimed in claim 1, wherein the porous particle has an oil absorption value in the range of 50 g/100 g to 500 g/100 g.

3. The personal care composition as claimed in claim 2, wherein the oil absorption value of the porous particle is 100 g/100 g to 300 g/100 g.

4. The personal care composition as claimed in claim 1, wherein the porous particle is present in amount of 0.1% to 10% by weight of the composition.

5. The personal care composition as claimed in claim 4, wherein the pore volume of the porous particle is in the range of 0.05 m³/g to 5 m³/g.

6. The personal care composition as claimed in claim 1, wherein the surface area of the porous particle is in the range of 50 m²/g to 500 m²/g.

7. The personal care composition as claimed in claim 6, wherein the surface area of the porous particle is in the range of 150 m²/g to 500 m²/g.

8. The personal care composition as claimed in claim 1, wherein the porous particle is present in amount of 1% to 8% by weight of the composition.

9. The personal care composition as claimed in claim 1, wherein the porous particle is porous silica.

10. The personal care composition as claimed in claim 1, wherein the weight ratio of the crosspolymer to the porous particle is in the range of 200:1 to 1:2.

11. The personal care composition as claimed in claim 1, wherein the crosspolymer comprising adipic acid and neopentyl glycol is present in the range of 5% to 50% by weight of the composition.

12. A method of improving the appearance of skin by blurring the imperfections of the skin comprising the steps of applying a personal care composition as claimed in claim 1 on the desired skin surface.

13. (canceled)

14. The method as claimed in claim 12, wherein the blurring of imperfections of the skin is provided for by reducing the appearance of fine lines, wrinkles, pores, and/or blemish spots on the skin.

15. The personal care composition as claimed in claim 1, wherein the weight ratio of the crosspolymer to the porous particle is in the range of 60:1 to 2:1.

16. The personal care composition as claimed in claim 1, wherein the weight ratio of the crosspolymer to the porous particle is in the range of 20:1 to 8:1.

17. The personal care composition as claimed in claim 1, wherein the crosspolymer comprising adipic acid and neopentyl glycol is present in the range of 12% to 42% by weight of the composition.
